(19) United States
(12) Patent Application Publication
(10) Pub. No.: US 2024/0377326 A1
(43) Pub. Date: Nov. 14, 2024

(54) USE OF FLUORESCENT OR VISIBLE TRACERS TO MONITOR NUTRIENT CONCENTRATIONS IN SOLUTION

(71) Applicant: FEHR SOLUTIONS, LLC, Geneva, IL (US)

(72) Inventor: Michael FEHR, Geneva, IL (US)

(21) Appl. No.: 18/692,732

(22) PCT Filed: Sep. 16, 2022
(86) PCT No.: PCT/US2022/043756

§ 371 Date: Mar. 15, 2024

Related U.S. Application Data
(60) Provisional application No. 63/245,311, filed on Sep. 17, 2021.

(51) Int. Cl. G01N 21/64; G01N 33/18

(52) U.S. Cl. CPC G01N 21/6428; G01N 33/18; G01N 2021/6441

(57) ABSTRACT

A method to determine whether the desired amount of at least two nutrient products is present in water, wherein the water is used to deliver nutrients to growing plants; is described and claimed. Inert fluorescent tracers are used proportionally to nutrients and then fluorometers are used to determine how much of the tracer is present, and from knowing that, the amount of nutrient present can be determined.

### FIELD OF INVENTION

This Invention is in the field of monitoring the concentration of nutrients applied to growing plants.

### BACKGROUND OF THE INVENTION

Plants can be grown in many different types of growth media. These growth media include, dirt, water and variations thereof.

Horticulture is the art of cultivating plants in gardens to produce food and medicinal ingredients, or for comfort and ornamental purposes. Horticulturists are agriculturists who grow flowers, fruits and nuts, vegetables and herbs, as well as ornamental trees and lawns.

Traditional Horticulture involves the planting of seed within dirt under adequate conditions of hydration and nutrition such that the seed will grow into the plant that eventually will be harvested. Other types of horticulture exist that are grouped into the category “soil-free” horticulture.

Hydroponics is a type of horticulture and a subset of hydroculture which involves growing plants (usually crops) without soil, by using mineral nutrient solutions in an aqueous solvent. Terrestrial plants may grow with their roots exposed to the nutritious liquid, or, in addition, the roots may be physically supported by an inert medium such as perlite, gravel, or other substrates. Despite inert media, roots can cause changes of the rhizosphere pH and root exudates can affect rhizosphere biology.

The nutrients used in hydroponic systems can come from many different sources, including fish excrement, duck manure, purchased chemical fertilizers, or artificial nutrient solutions.

Plants commonly grown hydroponically, on inert media, include tomatoes, peppers, cucumbers, strawberries, lettuces, cannabis, and model plants like Arabidopsis thaliana.

Hydroponics offers many advantages, notably a decrease in water usage in agriculture. To grow 1 kilogram (2.2 lb) of tomatoes using intensive farming methods requires 400 liters (88 imp gal; 110 U.S. gal) of water; using hydroponics, 70 liters (15 imp gal; 18 U.S. gal); and only 20 liters (4.4 imp gal; 5.3 U.S. gal) using aeroponics. Since hydroponics takes much less water to grow produce, it could be possible in the future for people in harsh environments with little accessible water to grow their own food.

### Passive Hydroponics

Passive sub-irrigation, also known as passive hydroponics, semi-hydroponics, or hydroculture, is a method wherein plants are grown in an inert porous medium that transports water and fertilizer to the roots by capillary action from a separate reservoir as necessary, reducing labor and providing a constant supply of water to the roots. In the simplest method, the pot sits in a shallow solution of fertilizer and water or on a capillary mat saturated with nutrient solution. The various hydroponic media available, such as expanded clay and coconut husk, contain more air space than more traditional potting mixes, delivering increased oxygen to the roots, which is important in epiphytic plants such as orchids and bromeliads, whose roots are exposed to the air in nature. Additional advantages of passive hydroponics are the reduction of root rot and the additional ambient humidity provided through evaporations.

Hydroculture compared to traditional soil-based horticulture in terms of crops yield per area in a controlled environment was roughly 10 times more efficient than traditional farming, uses 13 times less water in one crop cycle than traditional farming, but on average uses 100 times more kilojoules per kilogram of energy than traditional farming.

### Ebb and Flow (Flood and Drain) Sub-Irrigation

In its simplest form, there is a tray above a reservoir of nutrient solution. Either the tray is filled with growing medium (clay granules being the most common) and then plant directly or place the pot over medium, stand in the tray. At regular intervals, a simple timer causes a pump to fill the upper tray with nutrient solution, after which the solution drains back down into the reservoir. This keeps the medium regularly flushed with nutrients and air. Once the upper tray fills past the drain stop, it begins recirculating the water until the timer turns the pump off, and the water in the upper tray drains back into the reservoirs.

### Run-to-Waste

In a run-to-waste system, nutrient and water solution is periodically applied to the medium surface. The method was invented in Bengal in 1946; for this reason it is sometimes referred to as “The Bengal System”. This method can be set up in various configurations. In its simplest form, a nutrient-and-water solution is manually applied one or more times per day to a container of inert growing media, such as rockwool, perlite, vermiculite, coco fiber, or sand. In a slightly more complex system, it is automated with a delivery pump, a timer and irrigation tubing to deliver nutrient solution with a delivery frequency that is governed by the key parameters of plant size, plant growing stage, climate, substrate, and substrate conductivity, pH, and water content.

In a commercial setting, watering frequency is multi-factorial and governed by computers or PLCs.

Commercial hydroponics production of large plants like tomatoes, cucumber, and peppers uses one form or another of run-to-waste hydroponics.

Some bonsai are also grown in soil-free substrates (typically consisting of akadama, grit, diatomaceous earth and other inorganic components) and have their water and nutrients provided in a run-to-waste form.

### Deep Water Culture

The hydroponic method of plant production by means of suspending the plant roots in a solution of nutrient-rich, oxygenated water. Traditional methods favor the use of plastic buckets and large containers with the plant contained in a net pot suspended from the centre of the lid and the roots suspended in the nutrient solution. The solution is oxygen saturated by an air pump combined with porous stones. With this method, the plants grow much faster because of the high amount of oxygen that the roots receive. The Kratky Method is similar to deep water culture, but uses a non-circulating water reservoir.

### Top-Fed Deep Water Culture

Top-fed deep water culture is a technique involving delivering highly oxygenated nutrient solution direct to the root zone of plants. While deep water culture involves the plant roots hanging down into a reservoir of nutrient solution, in top-fed deep water culture the solution is pumped from the reservoir up to the roots (top feeding). The water is released over the plant's roots and then runs back into the reservoir below in a constantly recirculating system. As with deep water culture, there is an airstone in the reservoir that pumps air into the water via a hose from outside the reservoir. The airstone helps add oxygen to the water. Both the airstone and the water pump run 24 hours a day.

The biggest advantage of top-fed deep water culture over standard deep water culture is increased growth during the first few weeks. With deep water culture, there is a time when the roots have not reached the water yet. With top-fed deep-water culture, the roots get easy access to water from the beginning and will grow to the reservoir below much more quickly than with a deep water culture system. Once the roots have reached the reservoir below, there is not a huge advantage with top-fed deep-water culture over standard deep water culture. However, due to the quicker growth in the beginning, grow time can be reduced by a few weeks.

### Rotary

A rotary hydroponic garden is a style of commercial hydroponics created within a circular frame which rotates continuously during the entire growth cycle of whatever plant is being grown.

While system specifics vary, systems typically rotate once per hour, giving a plant 24 full turns within the circle each 24-hour period. Within the center of each rotary hydroponic garden can be a high intensity grow light, designed to simulate sunlight, often with the assistance of a mechanized timer.

Each day, as the plants rotate, they are periodically watered with a hydroponic growth solution to provide all nutrients necessary for robust growth. Due to the plants continuous fight against gravity, plants typically mature much more quickly than when grown in soil or other traditional hydroponic growing systems. Because rotary hydroponic systems have a small size, it allows for more plant material to be grown per area of floor space than other traditional hydroponic systems.

### Inorganic Hydroponic Solutions

The formulation of hydroponic solutions is an application of plant nutrition, with nutrient deficiency symptoms mirroring those found in traditional soil based agriculture. However, the underlying chemistry of hydroponic solutions can differ from soil chemistry in many significant ways. Important differences include:

Unlike soil, hydroponic nutrient solutions do not have cation-exchange capacity (CEC) from clay particles or organic matter. The absence of CEC and soil pores means the pH, oxygen saturation, and nutrient concentrations can change much more rapidly in hydroponic setups than is possible in soil.

Selective absorption of nutrients by plants often imbalances the amount of counterions in solution. This imbalance can rapidly affect solution pH and the ability of plants to absorb nutrients of similar ionic charge (see article membrane potential). For instance, nitrate anions are often consumed rapidly by plants to form proteins, leaving an excess of cations in solution. This cation imbalance can lead to deficiency symptoms in other cation based nutrients (e.g. Mg²⁺) even when an ideal quantity of those nutrients are dissolved in the solution.

Depending on the pH or on the presence of water contaminants, nutrients such as iron can precipitate from the solution and become unavailable to plants. Routine adjustments to pH, buffering the solution, or the use of chelating agents is often necessary.

Unlike soil types, which can vary greatly in their composition, hydroponic solutions are often standardized and require routine maintenance for plant cultivation. Hydroponic solutions are periodically pH adjusted to near neutral (pH≈6.0) and are aerated with oxygen. Also, water levels must be refilled to account for transpiration losses and nutrient solutions require re-fortification to correct the nutrient imbalances that occur as plants grow and deplete nutrient reserves. Sometimes the regular measurement of nitrate ions is used as a parameter to estimate the remaining proportions and concentrations of other nutrient ions in a solution.

As in conventional agriculture, nutrients should be adjusted to satisfy Liebig's law of the minimum for each specific plant variety. Liebig's law of the minimum, is a law about the growth of plants. Its states that the growth of a plant is limited by the resource, which is most scarce, and not by the total amount of resources available.

Nevertheless, generally acceptable concentrations for nutrient solutions exist, with minimum and maximum concentration ranges for most plants being somewhat similar. Most nutrient solutions are mixed to have concentrations between 1,000 and 2,500 ppm. Acceptable concentrations for the individual nutrient ions, which comprise that total ppm figure, are summarized in the following table. For essential nutrients, concentrations below these ranges often lead to nutrient deficiencies while exceeding these ranges can lead to nutrient toxicity. Optimum nutrition concentrations for plant varieties are found empirically by experience or by plant tissue tests.

### Organic Hydroponic Solutions

Organic fertilizers can be used to supplement or entirely replace the inorganic compounds used in conventional hydroponic solutions. However, using organic fertilizers introduces a number of challenges that are not easily resolved. Examples include:
organic fertilizers are highly variable in their nutritional compositions in terms of minerals and different chemical species. Even similar materials can differ significantly based on their source (e.g. the quality of manure varies based on an animal's diet).
organic fertilizers are often sourced from animal byproducts, making disease transmission a serious concern for plants grown for human consumption or animal forage.
organic fertilizers are often particulate and can clog substrates or other growing equipment. Sieving or milling the organic materials to fine dusts is often necessary.
some organic materials (i.e. particularly manures and offal) can further degrade to emit foul odors under anaerobic conditions.
many organic molecules (i.e. sugars) demand additional oxygen during aerobic degradation, which is essential for cellular respiration in the plant roots.
organic compounds are not necessary for normal plant nutrition.

Nevertheless, if precautions are taken, organic fertilizers can be used successfully in hydroponics.

### Common Equipment

Managing nutrient concentrations, oxygen saturation, and pH values within acceptable ranges is essential for successful hydroponic horticulture. Common tools used to manage hydroponic solutions include:
Electrical conductivity meters, a tool which estimates nutrient ppm by measuring how well a solution transmits an electric current.
pH meter, a tool that uses an electric current to determine the concentration of hydrogen ions in solution.
Oxygen electrode, an electrochemical sensor for determining the oxygen concentration in solution.
Litmus paper, disposable pH indicator strips that determine hydrogen ion concentrations by color changing chemical reaction.
Graduated cylinders or measuring spoons to measure out premixed, commercial hydroponic solutions.

### Equipment

Chemical equipment can also be used to perform accurate chemical analyses of nutrient solutions. Examples include:
Balances for accurately measuring materials.
Laboratory glassware, such as burettes and pipettes, for performing titrations.
Colorimeters for solution tests which apply the Beer-Lambert law.
Spectrophotometer to measure the concentrations of the lead parameter nitrate and other nutrients, such as phosphate, sulfate or iron.

Using chemical equipment for hydroponic solutions can be beneficial to growers of any background because nutrient solutions are often reusable. Because nutrient solutions are virtually never completely depleted, and should never be due to the unacceptably low osmotic pressure that would result, re-fortification of old solutions with new nutrients can save growers money and can control point source pollution, a common source for the eutrophication of nearby lakes and streams.

Aeroponics is a system wherein roots are continuously or discontinuously kept in an environment saturated with fine drops (a mist or aerosol) of nutrient solution. The method requires no substrate and entails growing plants with their roots suspended in a deep air or growth chamber with the roots periodically wetted with a fine mist of atomized nutrients. Excellent aeration is the main advantage of aeroponics.

Aeroponic techniques have proven to be commercially successful for propagation, seed germination, seed potato production, tomato production, leaf crops, and micro-greens. Since inventor Richard Stoner commercialized aeroponic technology in 1983, aeroponics has been implemented as an alternative to water intensive hydroponic systems worldwide. The limitation of hydroponics is the fact that 1 kilogram (2.2 lb) of water can only hold 8 milligrams (0.12 gr) of air, no matter whether aerators are utilized or not.

Another distinct advantage of aeroponics over hydroponics is that any species of plants can be grown in a true aeroponic system because the microenvironment of an aeroponic can be finely controlled. The limitation of hydroponics is that certain species of plants can only survive for so long in water before they become waterlogged. The advantage of aeroponics is that suspended aeroponic plants receive 100% of the available oxygen and carbon dioxide to the roots zone, stems, and leaves, thus accelerating biomass growth and reducing rooting times. NASA research has shown that growth and reducing rooting times. NASA research has shown that aeroponically grown plants have an 80% increase in dry weight biomass (essential minerals) compared to hydroponically grown plants. Aeroponics used 65% less water than hydroponics. NASA also concluded that aeroponically grown plants require % the nutrient input compared to hydroponics.

Unlike hydroponically grown plants, aeroponically grown plants will not suffer transplant shock when transplanted to soil and offers growers the ability to reduce the spread of disease and pathogens. Aeroponics is also widely used in laboratory studies of plant physiology and plant pathology. Aeroponic techniques have been given special attention from NASA since a mist is easier to handle than a liquid in a zero-gravity environment.

Fogponics is a derivation of aeroponics wherein the nutrient solution is aerosolized by a diaphragm vibrating at ultrasonic frequencies. Solution droplets produced by this method tend to be 5-10 μm in diameter, smaller than those produced by forcing a nutrient solution through pressurized nozzles, as in aeroponics. The smaller size of the droplets allows them to diffuse through the air more easily and deliver nutrients to the roots without limiting their access to oxygen.

### Nutrients

The vast majority of plants typically require three nutrients in large quantities. These macronutrients are nitrogen (“N”), phosphorus (“P”), and potassium (“K”), and they form the cornerstone of all plant health. As such, these three nutrients usually are recited prominently on the labels for fertilizer products in the form of the “NPK ratio”. The higher the number for each value, the higher the concentration of that particular nutrient.

Plants generally need more than just three nutrients to survive and thrive. Plants also count on secondary nutrients like calcium, magnesium, and sulfur to play vital roles in plant growth.

Calcium is important for cell wall development, can help reduce soil salinity, and improves water penetration when used as a soil amendment.

Magnesium plays a key role in photosynthesis and carbohydrate metabolism and helps with the stabilization of plant cell walls.

Sulfur is necessary for the formation of chlorophyll and the production of proteins, amino acids, enzymes, and vitamins, and protects plants against disease.

Plants also make use of several other nutrients in small quantities (“micronutrients”) that are nevertheless extremely important. These micronutrients include boron (“B”), chlorine (“Cl”), copper (“Cu”), iron(“Fe”), manganese (“Mn”), molybdenum (“Mo”) and zinc (“Zn”). Admittedly, although these micronutrients are not the main nutrients plants use for food, they still play significant roles in various aspects of plant health.

Most plant nutrients can be applied through irrigation systems. In environmentally responsible uses, the nutrient-rich waste of the irrigation system is collected and processed through an on-site filtration system to be used many times, making the system very productive.

Fertigation is the injection of fertilizers, used for soil amendments, water amendments and other water-soluble products into an irrigation system.

Fertigation is related to chemigation, the injection of chemicals into an irrigation system.

The two terms are sometimes used interchangeably however chemigation is generally a more controlled and regulated process due to the nature of the chemicals used. Chemigation often involves insecticides, herbicides, and fungicides, some of which pose health threat to humans, animals, and the environment.

Fertigation is practiced extensively in commercial agriculture and horticulture. Fertigation is also increasingly being used for landscaping as dispenser units become more reliable and easier to use. Fertigation is used to add additional nutrients or to correct nutrient deficiencies detected in plant tissue analysis. It is usually practiced on the high-value crops such as vegetables, turf, fruit trees, and ornamentals.

The benefits of fertigation methods over conventional or drop-fertilizing methods include, but are not limited to,
a) Increased nutrient absorption by plants;
b) Accurate placement of nutrient, where the water goes the nutrient goes as well;
c) Ability to “micro dose”, feeding the plants just enough so nutrients can be absorbed and are not left to be washed down to storm water next time it rains;
d) Reduction of fertilizer, chemicals, and water needed;
e) Reduced leaching of chemicals into the water supply;
f) Reduced water consumption due to the plant's increased root mass's ability to trap and hold water;
g) Application of nutrients can be controlled at the precise time and rate necessary;
h) Minimized risk of the roots contracting soil borne diseases through the contaminated soil;
i) Reduction of soil erosion issues as the nutrients are pumped through the water drip system; and
j) Leaching is decreased often through methods used to employ fertigation.

The disadvantages of fertigation include, but are not limited to:
a) the concentration of the solution may decrease as the fertilizer dissolves, this depends on equipment selection. If poorly selected may lead to poor nutrient placement;
b) The water supply for fertigation is to be kept separate from the domestic water supply to avoid contamination;
c) Possible pressure loss in the main irrigation line; and
d) The process is dependent on the water supply's non-restriction by drought rationing.

Irrigation and fertilizers (herein referred to collectively as fertigation) used in production of vegetables, fruits, and other plants relies on the proper ratios and levels of macro nutrients (N, P, K, Ca, Mg) as well as the proper levels of micronutrients (Fe, Cu, B, Mo . . . ). The exact ratios of these macros vary based on what is being produced, the life stage of the plant, and based on the cultivators' individual preferences and experiences. Regardless of the plant, it is important however to maintain consistent fertigation ratios and concentrations for proper plant health.

Currently, the most common way to control plant nutrient concentration and ratios is to purchase blended or individual nutrient systems from a variety of commercial suppliers (HGV, Jacks, Petes). These nutrients can come in either dry or liquid concentrated form or a mixture of both. Dry nutrients are dissolved in water—generally in 1 lb/gallon, 2 lb/gallon or 3 lb/gallon—to make concentrated batch tanks. The amount of dry nutrient in water varies from about 1 lb/US gallon, 2 lb/US gallon or 3 lb/US gallon. Liquid nutrients can also be prepared and diluted.

These concentrated nutrients are diluted with an acceptable water source (well water, city water, dehumidification condensate or reverse osmosis) to produce the desired levels and ratios of macro nutrients and micronutrients for fertigation. The dilution is based on a recipe that is created by the nutrient producers and/or the cultivator based on incoming water quality and the type of plant. One of the most common ways to measure and control this dilution is based on the Electrical Conductivity (“EC”), as reported in units of siemens per meter (S/m or mS/cm) of the resultant fertigation stream.

It is normal during the life cycle of a plant that its nutritional requirements change.

For the Cannabis plant the nutrient requirements change depending on the life stage.

### Nutrients for Cannabis Seedlings

Cannabis seedlings get all their nutrients from their seed and absorb water via their leaves as their root system develops (that is why it is important to keep them in a warm, humid environment).

Cannabis seedlings do not need nutritional supplements until they are about 3-4 weeks old, at which point they will have developed 3-4 true leaves, thus entering the vegetative growth phase.

### Nutrients for Vegetative Cannabis Plants

Some growers opt to start their plants off on a light 2:1:2 (ratio of N:P:K) in the fertilizer for one week just when their seedlings start to enter their vegetative growth phase. This step is a good way to introduce Cannabis plants to their fertilizer mixture and avoid nutrient burn.

Under certain conditions with certain plants, some growers see better results immediately starting their plants on a 4:2:3 (ratio of N:P:K) fertilizer to kickstart growth.

By the mid-vegetative phase (roughly 6 weeks after germinating), the plant nutritional additives need to increase in order to allow the plants to develop strong, healthy foliage. Most growers opt for a 10:5:7 (ratio of N:P:K) fertilizer by this stage. These heightened levels of nitrogen will help vegging Cannabis plants produce luscious, green foliage and develop plenty of bud sites in time for flowering. Towards the end of the vegetative phase, it is a good idea to start lowering the nitrogen levels and preparing the Cannabis plants for the switch to their bloom booster. Most growers use a 7:7:7 (ratio of N:P:K) fertilizer in the last week of the vegetative phase.

### Cannabis Vegetative Feeding Recommendations Through the Life Cycle of the Cannabis Plant:

Early veg: 2:1:2-4:2:3 (ratio of N:P:K)
Mid-veg: 10:5:7 (ratio of N:P:K)
Late veg: 7:7:7 (ratio of N:P:K)

### Nutrients for Flowering Cannabis Plants

Flowering cannabis plants need less nitrogen and more potassium to promote the growth of big, resinous flowers. During the first two weeks of flowering, most growers feed their flowering plants with a 5:7:10 (ratio of N:P:K) fertilizer. By mid-flowering, most growers will be using a 6:10:15 (ratio of N:P:K) nutrient solution.

During the last weeks of flowering, growers will drive down their nutrients to smooth out the transition to the pre-harvest flush. By this stage, it is common to use a milder fertilizer with an NPK ratio of 4:7:10.

### Cannabis Feeding Recommendations During the Flowering Stage:

Early bloom: 5:7:10
Mid-bloom: 6:10:15
Mid-late bloom: 4:7:10
Late bloom: pH balanced flush
Here is one type of Cannabis Formula useful during the flowering stage with a 1:4:0.4 NPK ratio.

TABLE 1

| Nutrient (as ppm) | ppm |
|---|---|
| N (NO₃_{^{−}}) | 141 |
| K | 433 |
| P | 96 |
| Mg | 120 |
| Ca | 186 |
| S | 158 |
| Fe | 6 |
| Zn | 0.3 |
| B | 1 |
| Cu | 0.3 |
| Mo | 0.2 |
| Na | 1.2 |
| Si | 0 |
| Cl | 0 |
| Mn | 1 |
| N (NH₄_{⁺}) | 10.8 |
| EC(mS/cm) | 2.496 |

The final EC of this solution in pure water (example—RO permeate) is 2.496.
If the source water EC is 0.300 the final EC would be 2.496+0.300=2.796 EC

Achieving these concentrations can be done in a variety of methods including but not limited to:
1) Weighing out individual quantities (as shown in the above formula). While this technique leads to exactly accurate results, this method is generally not done in practice as it is time consuming.
2) Using a combination of preblended dry or liquid nutrients such as
a. Calcium Nitrate (ag grade)
b. Peter's (5,11,25) where the numbers represent (P,K,N) expressed as percent P₂O₅, K₂O, and N.
   i. This formulation also includes the “micro” nutrients that are required such as (Fe, Cu, Mn, Mo, B, Zn . . . )
   ii. And can contain MgSO₄ (Epsom salts).
c. To make this formula two solutions of Calcium Nitrate (ag grade) and Peter's (5,11,25) would be prepared at a known concentration (1 lb/gallon for example).
d. Each solution would then be fed at a desired dilution rate (typically done with a positive displacement pump or venturi) to a stream or tank of water. Control of the dilution rate is done volumetrically (e.g. at X.X oz/gallon), percent dilution (0.XX %) and/or using EC.
e. The EC can be measured individually (after each nutrient is added) or as a “sum” of the nutrients.
   i. For example, if the incoming water EC is 0.300 the dilution of Calcium nitrate to provide the desired amount of Nitrogen is 1.00 so the resultant solution will have an EC of 1.300 (+/−based on accuracy of EC meter). The dilution of Peters (5, 11, 25) adds an additional 1.500 EC so the final solution will have an EC of 1.30+1.50=2.80 EC (+/−).
   ii. Placement of the EC meters can be done in a variety of ways to confirm addition of the nutrients.
   iii. While the Peter's example is a “2 nutrient” system, “3 nutrient systems” and “4 nutrient systems”, and so on are also used.
   iv.
      Once a batch tank of material has been made there are no easy individual signatures of the nutrients that can be quickly measured on-site without time consuming and laborious wet chemistry, ion selective electrodes or more advanced analytical techniques. Because fertigation solutions typically are used right after they are made (with some storage capacities) there is not time for each batch to be sent out for an accurate analysis.
      While direct measurement of the macro nutrient ions (P, K and N) could alleviate this issue at the present time, sensors that measure these ions in real time at typical concentrations are not readily available, cost effective or reliable.
      What is needed is a method to determine the amount of each nutrient ion that is fast, reliable and that can be done on site.

### SUMMARY OF THE INVENTION

The first aspect of the instant claimed invention is a method to optimize horticulture of growing plants by determining whether the desired amount of at least two nutrient products is present in water, wherein the water is used to deliver nutrients to growing plants; comprising the steps of:
a) adding a known amount of an inert fluorescent tracer to a known amount of a nutrient, wherein said nutrient is useful to provide nutrition to growing plants;
b) adding at least two or more mixtures of inert fluorescent tracer and nutrient to water to create a nutrient liquid; wherein the two or more mixtures have different fluorescent tracers and a different nutrient;
c) using two or more fluorometers to determine the concentration of two or more inert fluorescent materials in the water and using the concentration of two or more inert fluorescent tracers to calculate the amount of each nutrient present in the water;
d) adjusting the amount of nutrient present in the water when at least one of the fluorescent signals of at least one of the inert fluorescent tracers indicates either too much or too little of the nutrient matched with that inert fluorescent tracer is present; and
e) applying the nutrient liquid to growing plants.

The second aspect of the instant claimed invention is the method of the first aspect of the instant claimed invention, wherein the inert fluorescent material is selected from the group consisting of:
1,3,6,8-pyrenetetrasulfonic acid, tetrasodium salt (CAS Registry No. 59572-10-0);
1,5-naphthalenedisulfonic acid, disodium salt (hydrate) (CAS Registry No. 1655-29-4, aka 1,5-NDSA hydrate);
xanthylium, 9-(2,4-dicarboxyphenyl)-3,6-bis(diethylamino), chloride, disodium salt, also known as Rhodamine WT (CAS Registry No. 37299-86-8);
C.I. Fluorescent Brightener 230, also known as Leucophor BSB (CAS Registry No. 68444-86-0);
benzenesulfonic acid, 2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Leucophor BMB (CAS Registry No. 16470-249, aka Leucophor U, Flu. Bright. 290);
9,9′-biacridinium, 10,10′-dimethyl-, dinitrate, also known as Lucigenin (CAS Registry No. 2315-97-1, aka bis-N-methylacridinium nitrate);
1-deoxy-1-(3,4-dihydro-7,8-dimethyl-2,4-dioxobenzo[g]pteridin-10(2H)-yl)-D-ribitol, also known as Riboflavin or Vitamin B2 (CAS Registry No. 83-88-5);
3,6-acridinediamine, N,N,N′,N′-tetramethyl-, monohydrochloride, also known as Acridine Orange (CAS Registry No. 65-61-2);
2-anthracenesulfonic acid sodium salt (CAS Registry No. 16106-40-4);
1,5-anthracenedisulfonic acid (CAS Registry No. 61736-91-2) and salts thereof;
2,6-anthracenedisulfonic acid (CAS Registry No. 61736-95-6) and salts thereof;
1,8-anthracenedisulfonic acid (CAS Registry No. 61736-92-3) and salts thereof;
anthra[9,1,2-cde]benzo[rst]pentaphene-5,10-diol,16,17-dimethoxy-,bis(hydrogen sulfate), disodium salt, also known as Anthrasol Green IBA (CAS Registry No. 2538-84-3, aka Solubilized Vat Dye);
bathophenanthrolinedisulfonic acid disodium salt (CAS Registry No. 52746-49-3);
amino 2,5-benzene disulfonic acid (CAS Registry No. 41184-20-7);
2-(4-aminophenyl)-6-methylbenzothiazole (CAS Registry No. 92-364);
1H-benz[de]isoquinoline-5-sulfonic acid, 6-amino-2,3-dihydro-2-(4-methylphenyl)1,3-dioxo-, monosodium salt, also known as Brilliant Acid Yellow 8G (CAS Registry No. 2391-30-2, aka Lissamine Yellow FF, Acid Yellow 7);
phenoxazin-5-ium, 1-(aminocarbonyl)-7-(diethylamino)-3,4-dihydroxy-, chloride, also known as Celestine Blue (CAS Registry No. 1562-90-9);
benzo[a]phenoxazin-7-ium, 5,9-diamino-, acetate, also known as cresyl violet acetate (CAS Registry No. 10510-54-0);
4-dibenzofuransulfonic acid (CAS Registry No. 42137-76-8);
3-dibenzofuransulfonic acid (CAS Registry No. 215189-98-3);
1-ethylquinaldinium iodide (CAS Registry No. 606-53-3);
fluorescein (CAS Registry No. 2321-07-5);
fluorescein, sodium salt (CAS Registry No. 518-47-8, aka Acid Yellow 73, Uranine);
Keyfluor White ST (CAS Registry No. 144470-48-4, aka Flu. Bright 28);
Benzenesulfonicacid2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Keyfluor White CN (CAS Registry No. 16470-24-9);
pyranine, (CAS Registry No. 6358-69-6, aka 8-hydroxy-1, 3, 6-pyrenetrisulfonic acid, trisodium salt);
quinoline (CAS Registry No. 91-22-5);
3H-phenoxazin-3-one, 7-hydroxy-, 10-oxide, also known as Rhodalux (CAS Registry No. 550-82-3);
xanthylium, 9-(2,4-dicarboxyphenyl)-3,6-bis(diethylamino)-, chloride, disodium salt, also known as Rhodamine WT (CAS Registry No. 37299-86-8); and
phenazinium, 3,7-diamino-2,8-dimethyl-5-phenyl-, chloride, also known as Safranine 0 (CAS Registry No. 477-73-6).

The third aspect of the instant claimed invention is the method of the second aspect of the instant claimed invention, wherein the inert fluorescent tracer is selected from the group consisting of PTSA, NDSA and Rhodamine.

The fourth aspect of the instant claimed invention is the method of the first aspect of the instant claimed invention, wherein the nutrients present in the nutrient liquid include nitrogen, phosphorus, and potassium.

The fifth aspect of the instant claimed invention is the method of the third aspect of the instant claimed invention, wherein the inert fluorescent tracer for nitrogen is PTSA, the inert fluorescent tracer for phosphorus is NDSA and the inert fluorescent tracer for potassium is Rhodamine.

The sixth aspect of the instant claimed invention is the method of the first aspect of the instant claimed invention wherein the Horticulture methods are selected from the group consisting of Traditional Horticulture, Hydroponics, Passive hydroponics, Hydroculture, Deep water culture, Top-fed deep water culture, Rotary Hydroponic Garden, Inorganic hydroponic solutions, Organic hydroponic solutions, hydroponic solutions, Aeroponics, Fogponics and Fertigation.

The seventh aspect of the instant claimed invention is the method of the first aspect of the instant claimed invention wherein the growing plants are selected from the group consisting of fruits, vegetables, tobacco, Cannabis, Hemp and trees.

The eighth aspect of the instant claimed invention is the method of the seventh aspect of the instant claimed invention wherein the growing plants are selected from the group consisting of fruits.

The ninth aspect of the instant claimed invention is the method of the seventh aspect of the instant claimed invention wherein the growing plants are selected from the group consisting of vegetables.

The tenth aspect of the instant claimed invention is the method of the seventh aspect of the instant claimed invention wherein the growing plants are selected from the group consisting of tobacco, Cannabis and Hemp Plants.

The eleventh aspect of the instant claimed invention is the method of the seventh aspect of the instant claimed invention wherein the growing plants are tobacco.

The twelfth aspect of the instant claimed invention is the method of the seventh aspect of the instant claimed invention wherein the growing plants are Cannabis.

The thirteenth aspect of the instant claimed invention is the method of the seventh aspect of the instant claimed invention wherein the growing plants are Hemp.

The fourteenth aspect of the instant claimed invention is the method of the seventh aspect of the instant claimed invention wherein the growing plants are trees.

### DETAILED DESCRIPTION OF THE INVENTION

The amounts of nutrients can be determined by introducing fluorescent or visible tracers that are intentionally dosed into the concentrate tank in proportion to the amount of P, K and N (and/or other nutrients) in the solution. These fluorescent tracers can then be individually resolved for P, K and N (and/or other ions) as the fertigation tank or solution is produced to ensure that proper concentrations and ratios are maintained. Furthermore, the concentration of each macronutrient (or micronutrients) can be inferred anywhere in the system (storage tanks, drip system for plants, leachate or plant run off) to determine if the correct nutrient levels are being achieved.

Table 2 and 3—concentrations of P, K and N versus fluorescent tracers

As an example, setting the P, K and N concentrations as follows

TABLE 2

| Fluorescent | Nutrient Concentration | | Fluorescent Tracer | |
|---|---|---|---|---|
| Tracers | (ppm as element) | | Concentration | |
| PTSA | 100 | ppm as N | 0.1 | ppm as PTSA |
| NDSA | 100 | ppm as P | 0.1 | ppm as NDSA |
| Rhodamine | 100 | ppm K | 0.01 | ppm as Rhodamine |

From Table 1 the target concentrations in this nutrition formula are
141 ppm of N (from both Peters and Calcium nitrate)
96 ppm of P (from Peters)
433 ppm of K (from Peters)

Using the ratios above this would equate to the following final concentrations:

TABLE 4

| Fluorescent | Concentration | Equivalent concentration | |
|---|---|---|---|
| Tracer | (ppm) | of Nutrient | |
| PTSA | 0.141 | 141 | ppm of N |
| NDSA | 0.096 | 96 | ppm of P |
| Rhodamine | 0.043 | 433 | ppm of K |

Unlike what is taught in U.S. Pat. No. 10,765,999, “Treatment of Industrial Water Systems”, assigned to Ecolab USA Inc., Inventors: Narasimha M. Rao, Steven R. Hatch and William A. Von Drasek which is the use of only one tracer per liquid, the Peters nutritional supplement would be formulated with three distinct fluorescent tracers, so as to make possible the tracing of the “big three” nutrients. Meanwhile, separate from the Peters nutritional supplement, calcium nitrate would only have one fluorescent tracer present as it only supplies one nutrient, nitrogen.

Fertigation water is often prepared in storage tanks that represent the entire days fertigation volume but can be made in smaller or larger batches. Fertigation water can also be made without batch tanks and diluted and delivered directly to plants. Typically, fertigation water is made up of a main water stream to which various plant nutrients are added. After each addition of plant nutrient, a water sample can be tested by using a testing tube alternative route where a fluorometer analyses of the fluorescent signal of the chemicals in the water is done. This information can either be read manually by the operator of the fertigation system, or the fluorescent signal can be transmitted to a computer that automatically compiles, reports the data, and follows a preprogrammed response if the signal is too low or too high, indicating to little or too much of the nutrient is present, to adjust the amount of nutrient added to the fertigation water.

The benefits of this invention are clear. The first benefit is that using fluorometric analysis removes the uncertainty of the variability of the EC in the source water. The source water often can have some EC (0.300 in the write-up above). If the source water EC changes to 0.500 and the final nutrient EC is set at 2.796 the nutrient dosages will not be correct or will need to be adjusted. Fluorometric analysis is not affected by the EC of the source water.

Measuring the tracer and obtaining the concentration of nutrient removes the need to accurately weigh out the nutrient. Feed systems (either venturis or pumps) can automatically adjust to changes in stock solution concentrations if the pump system is designed for this variability of delivery volume (e.g., 0.5 to 3 lbs/gallon mixtures). This aspect of the invention would reduce and/or eliminate a large source of error in most feed systems.

Accurate measurement of nutrient concentration can be done in tanks, at the plant, or in the piping by using either a handheld fluorometer or inline fluorometer. These results could be used to control dosage pumps (to make the solution), to monitor and report the concentration of dyes in tanks or within grow rooms (at the point of entry into the plant) and measure the nutrient post plant (as it leaves the media). This data can be trended and alarmed as desired by the cultivator.

Another positive feature of the instant invention is the excess nutrient (the nutrient that is expelled from the bottom of the media) is mixed with irrigation water as well as other nutrient blends (often called “veg” for vegetative and “bloom” when flowering is initiated). These streams are often mixed along with irrigation water and referred to as “leachate”. The nutrient levels can be significant in leachate streams, and it is desirable to reuse them. Positive aspects of reusing the leachate are that it both reduces costs by reducing nutrient usage and improves sustainability by preventing nutrients from being discharged to wastewater treatment plants or other sources where they end up requiring remediation through biological treatment facilities (adding load) or impact natural bodies of water. At present, certain states are requiring leachate reuse to prevent loading of nutrients into wastewater streams because in certain locations up to about 75% of the nutrient often goes down the drain.

Tracers used in the instant claimed process have the following properties:

Stable fluorescent or absorbance signal over the normal pH range within the nutrient systems (typically a pH of 5.0 to 7.0).

Fluorescent or absorbance-based tracers should mimic nutrient movement within the fertigation system including
Similar uptake by plant;
Similar interaction with growth media;
Similar interaction with other additives;
Not degrade due to UV light; and be individually resolvable through normal spectrophotometric or fluorescent techniques (filters, gratings, light sources).

A variety of different and suitable types of compounds can be used as suitable inert fluorescent tracers. In an embodiment, the inert fluorescent compounds are selected from the group consisting of the following compounds:
1,3,6,8-pyrenetetrasulfonic acid (also known as “PTSA”), tetrasodium salt (CAS Registry No. 59572-10-0);
1,5-naphthalenedisulfonic acid, disodium salt (hydrate) (CAS Registry No. 1655-29-4, aka 1,5-NDSA hydrate, aka NDSA);
xanthylium, 9-(2,4-dicarboxyphenyl)-3,6-bis(diethylamino), chloride, disodium salt, also known as Rhodamine WT (CAS Registry No. 37299-86-8);
3,6-acridinediamine, N,N,N′,N′-tetramethyl-, monohydrochloride, also known as Acridine Orange (CAS Registry No. 65-61-2);
2-anthracenesulfonic acid sodium salt (CAS Registry No. 16106-40-4);
1,5-anthracenedisulfonic acid (CAS Registry No. 61736-91-2) and salts thereof;
2,6-anthracenedisulfonic acid (CAS Registry No. 61736-95-6) and salts thereof;
1,8-anthracenedisulfonic acid (CAS Registry No. 61736-92-3) and salts thereof;
anthra[9,1,2-cde]benzo[rst]pentaphene-5, 10-diol, 16,17-dimethoxy-, bis(hydrogen sulfate), disodium salt, also known as Anthrasol Green IBA (CAS Registry No. 2538-84-3, aka Solubilized Vat Dye);
bathophenanthrolinedisulfonic acid disodium salt (CAS Registry No. 52746-49-3);
amino 2,5-benzene disulfonic acid (CAS Registry No. 41184-20-7);
2-(4-aminophenyl)-6-methylbenzothiazole (CAS Registry No. 92-364);
1H-benz[de]isoquinoline-5-sulfonic acid, 6-amino-2,3-dihydro-2-(4-methylphenyl)1,3-dioxo-, monosodium salt, also known as Brilliant Acid Yellow 8G (CAS Registry No. 2391-30-2, aka Lissamine Yellow FF, Acid Yellow 7);
phenoxazin-5-ium, 1-(aminocarbonyl)-7-(diethylamino)-3,4-dihydroxy-, chloride, also known as Celestine Blue (CAS Registry No. 1562-90-9);
benzo[a]phenoxazin-7-ium, 5,9-diamino-, acetate, also known as cresyl violet acetate (CAS Registry No. 10510-54-0);
4-dibenzofuransulfonic acid (CAS Registry No. 42137-76-8);
3-dibenzofuransulfonic acid (CAS Registry No. 215189-98-3);
1-ethylquinaldinium iodide (CAS Registry No. 606-53-3);
fluorescein (CAS Registry No. 2321-07-5);
fluorescein, sodium salt (CAS Registry No. 518-47-8, aka Acid Yellow 73, Uranine);
Keyfluor White ST (CAS Registry No. 144470-48-4, aka Flu. Bright 28);
benzenesulfonic acid, 2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Keyfluor White CN (CAS Registry No. 16470-24-9);
C.I. Fluorescent Brightener 230, also known as Leucophor BSB (CAS Registry No. 68444-86-0);
benzenesulfonic acid, 2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Leucophor BMB (CAS Registry No. 16470-249, aka Leucophor U, Flu. Bright. 290);
9,9′-biacridinium, 10,10′-dimethyl-, dinitrate, also known as Lucigenin (CAS Registry No. 2315-97-1, aka bis-N-methylacridinium nitrate);
1-deoxy-1-(3,4-dihydro-7,8-dimethyl-2,4-dioxobenzo[g]pteridin-10(2H)-yl)-D-ribitol, also known as Riboflavin or Vitamin B2 (CAS Registry No. 83-88-5);
mono-, di-, or tri-sulfonated napthalenes, including but not limited to
2-amino-1-naphthalenesulfonic acid (CAS Registry No. 81-16-3);
5-amino-2-naphthalenesulfonic acid (CAS Registry No. 119-79-9);
4-amino-3-hydroxy-1-naphthalenesulfonic acid (CAS Registry No. 90-51-7);
6-amino-4-hydroxy-2-naphthalenesulfonic acid (CAS Registry No. 116-63-2);
7-amino-1,3-naphthalenesulfonic acid, potassium salt (CAS Registry No. 79873-35-1);
4-amino-5-hydroxy-2,7-naphthalenedisulfonic acid (CAS Registry No. 90-20-0);
5-dimethylamino-1-naphthalenesulfonic acid (CAS Registry No. 4272-77-9);
1-amino-4-naphthalene sulfonic acid (CAS Registry No. 84-86-6);
1-amino-7-naphthalene sulfonic acid (CAS Registry No. 119-28-8); and
2,6-naphthalenedicarboxylic acid, dipotassium salt (CAS Registry No. 2666-06-0);
3,4,9,10-perylenetetracarboxylic acid (CAS Registry No. 81-32-3);
C.I. Fluorescent Brightener 191, also known as Phorwite CL (CAS Registry No. 12270-53-0);
C.I. Fluorescent Brightener 200, also known as Phorwite BKL (CAS Registry No. 61968-72-7);
benzenesulfonic acid, 2,2′-(1,2-ethenediyl)bis[5-(4-phenyl-2H-1,2,3-triazol-2-yl)-, dipotassium salt, also known as Phorwite BHC 766 (CAS Registry No. 52237-03-3);
benzenesulfonic acid, 5-(2H-naphtho[1,2-d]triazol-2-yl)-2-(2-phenylethenyl)-, sodium salt, also known as Pylaklor White S—ISA (CAS Registry No. 6416-68-8);
pyranine, (CAS Registry No. 6358-69-6, aka 8-hydroxy-1, 3, 6-pyrenetrisulfonic acid, trisodium salt);
quinoline (CAS Registry No. 91-22-5);
3H-phenoxazin-3-one, 7-hydroxy-, 10-oxide, also known as Rhodalux (CAS Registry No. 550-82-3);
phenazinium, 3,7-diamino-2,8-dimethyl-5-phenyl-, chloride, also known as Safranine 0 (CAS Registry No. 477-73-6);
C.I. Fluorescent Brightener 235, also known as Sandoz CW (CAS Registry No. 56509-06-9);
benzenesulfonic acid, 2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Sandoz CD (CAS Registry No. 16470-24-9, aka Flu. Bright. 220);
benzenesulfonic acid, 2,2′-(1,2-ethenediyl)bis[5-[[4-[(2-hydroxypropyl)amino]-6-(phenylamino)-1, 3,5-triazin-2-yl]amino]-, disodium salt, also known as Sandoz TH-40 (CAS Registry No. 32694-95-4);
xanthylium, 3,6-bis(diethylamino)-9-(2,4-disulfophenyl)-, inner salt, sodium salt, also known as Sulforhodamine B (CAS Registry No. 3520-42-1, aka Acid Red 52);
benzenesulfonic acid, 2,2′-(1,2-ethenediyl)bis[5-[[4-[(aminomethylx2-hydroxyethyl)amino]-6-(phenylamino)-1,3,5-triazin-2-yl]amino]-, disodium salt, also known as Tinopal 5BM-GX (CAS Registry No. 169762-28-1);
Tinopol DCS (CAS Registry No. 205265-33-4);
benzenesulfonic acid, 2,2′-([1,1′-biphenyl]-4,4′-diyldi-2,1-ethenediyl)bis-, disodium salt, also known as Tinopal CBS-X (CAS Registry No. 27344-41-8);
benzenesulfonic acid, 5-(2H-naphtho[1,2-d]triazol-2-yl)2-(2-phenylethenyl)-, sodium salt, also known as Tinopal RBS 200, (CAS Registry No. 6416-68-8);
7-benzothiazolesulfonic acid, 2,2′-(1-triazene-1,3-diyldi-4,1-phenylene)bis[6-methyl-, disodium salt,
also known as Titan Yellow (CAS Registry No. 1829-00-1, aka Thiazole Yellow G); and all ammonium, potassium, and sodium salts thereof, and all like agents and suitable mixtures thereof.

In an embodiment, inert fluorescent tracers useful in the method of the present invention include
1,3,6,8-pyrenetetrasulfonic acid tetrasodium salt (CAS Registry No. 59572-10-0);
1,5-naphthalenedisulfonic acid disodium salt (hydrate) (CAS Registry No. 1655-29-4, aka 1,5-NDSA hydrate);
xanthylium, 9-(2,4-dicarboxyphenyl)-3,6-bis(diethylamino), chloride, disodium salt, also known as Rhodamine WT (CAS Registry No. 37299-86-8);
1-deoxy-1-(3,4-dihydro-7,8-dimethyl-2,4-dioxobenzo[g]pteridin-10(2H)-yl)-D-ribitol, also known as Riboflavin or Vitamin B2 (CAS Registry No. 83-88-5);
fluorescein (CAS Registry No. 2321-07-5);
fluorescein, sodium salt (CAS Registry No. 518-47-8, aka Acid Yellow 73, Uranine);
2-anthracenesulfonic acid sodium salt (CAS Registry No. 16106-40-4);
2,6-anthracenedisulfonic acid (CAS Registry No. 61736-95-6) and salts thereof;
1,8-anthracenedisulfonic acid (CAS Registry No. 61736-92-3) and salts thereof; and mixtures thereof.

In an embodiment, the fluorescent tracers are selected from the group consisting of PTSA, NDSA and Rhodamine WT.

The fluorescent tracers listed above are commercially available from a variety of different chemical supply companies.

Flourometers are commercially available from Pyxis and Turner Designs among other equipment manufacturers.

Fruits and Vegetables that can Make Use of this Process

In botany, a fruit is the seed-bearing structure in flowering plants that is formed from the ovary after flowering.

Fruits are the means by which flowering plants (also known as angiosperms) disseminate their seeds. Edible fruits in particular have long propagated using the movements of humans and animals in a symbiotic relationship that is the means for seed dispersal for the one group and nutrition for the other; in fact, humans and many animals have become dependent on fruits as a source of food. Consequently, fruits account for a substantial fraction of the world's agricultural output, and some (such as the apple and the pomegranate) have acquired extensive cultural and symbolic meanings.

In common language usage, “fruit” normally means the fleshy seed-associated structures (or produce) of plants that typically are sweet or sour and edible in the raw state, such as apples, bananas, grapes, lemons, oranges, and strawberries. In botanical usage, the term “fruit” also includes many structures that are not commonly called “fruits”, such as nuts, bean pods, corn kernels, tomatoes, and wheat grains.

Types of dry simple fruits, include, but are not limited to:
achene—most commonly seen in aggregate fruits—for example strawberry.
capsule—Brazil nut: botanically, it is not a nut.
caryopsis—cereal grains, including wheat, rice, oats, barley.
cypsela—an achene-like fruit derived from the individual florets in a capitulum: (dandelion).
fibrous drupe—coconut, walnut: botanically, neither is a true nut.
follicle—a follicles fruit is formed from a single carpel, and opens by one suture: (milkweed); also commonly seen in aggregate fruits: (magnolia, peony).
legume—bean, pea, peanut: botanically, the peanut is the seed of a legume, not a nut.
loment—a type of indehiscent legume: (sweet vetch or wild potato).
nut—beechnut, hazelnut, acorn (of the oak): botanically, these are true nuts.
samara—ash, elm, maple key.
schizocarp—carrot seed.
silique—radish seed.
silicle—shepherd's purse.
utricle—strawberry.

Berries are a type of simple fleshy fruit that issue from a single ovary.[(The ovary itself may be compound, with several carpels.) The botanical term “true berry” includes grapes, currants, cucumbers, eggplants (aubergines), tomatoes, chili peppers, and bananas, but excludes certain fruits that are called “-berry” by culinary custom or by common usage of the term—such as strawberries and raspberries. Berries may be formed from one or more carpels (i.e., from the simple or compound ovary) from the same, single flower. Seeds typically are embedded in the fleshy interior of the ovary.

Examples of berries, include, but are not limited to:
tomato—In culinary terms, the tomato is regarded as a vegetable, but it is botanically classified as a fruit and a berry.
banana—The fruit has been described as a “leathery berry”. In cultivated varieties, the seeds are diminished nearly to non-existence.
pepo—Berries with skin that is hardened: cucurbits, including gourds, squash, melons.
hesperidium—Berries with a rind and a juicy interior: most citrus fruit.
cranberry, gooseberry, redcurrant, grape.

Schizocarps are dry fruits, though some appear to be fleshy. They originate from syncarpous ovaries but do not actually dehisce; rather, they split into segments with one or more seeds. They include a number of different forms from a wide range of families, including carrot, parsnip, parsley, cumin.

Types of fleshy simple fruits, (with examples) include, but are not limited to:
berry—The berry is the most common type of fleshy fruit. The entire outer layer of the ovary wall ripens into a potentially edible “pericarp”.
stone fruit or drupe—The definitive characteristic of a drupe is the hard, “lignified” stone (sometimes called the “pit”). It is derived from the ovary wall of the flower: apricot, cherry, olive, peach, plum, mango.
pome—The pome fruits: apples, pears, rosehips, saskatoon berry, et al., are a syncarpous (fused) fleshy fruit, a simple fruit, developing from a half-inferior ovary,
Pomes are of the family Rosaceae,

### Types of Fleshy Fruits

| Type | Examples |
|---|---|
| Simple fleshy fruit | True berry, Stone fruit, Pome |
| Aggregate fruit | Boysenberry, Lilium, Magnolia, Raspberry, |
| | Pawpaw, Blackberry, Strawberry |
| Multiple fruit | Fig, Osage orange, Mulberry, Pineapple |
| True berry | Banana, Blackcurrant, Blueberry, Chili |
| | pepper, Cranberry, Eggplant, Gooseberry, |
| | Grape, Guava, Kiwifruit, Lucuma, Pomegranate, |
| | Redcurrant, Tomato, Watermelon |
| True berry: Pepo | Cucumber, Gourd, Melon, Pumpkin |
| True berry: | Grapefruit, Lemon, Lime, Orange |
| Hesperidium | |
| Accessory fruit | Apple, Rose hip, Stone |
| | fruit, Pineapple, Blackberry, Strawberry |

Vegetables are parts of plants that are consumed by humans or other animals as food. The original meaning is still commonly used and is applied to plants collectively to refer to all edible plant matter, including the flowers, fruits, stems, leaves, roots, and seeds. An alternate definition of the term is applied somewhat arbitrarily, often by culinary and cultural tradition. It may exclude foods derived from some plants that are fruits, flowers, nuts, and cereal grains, but include savory fruits such as tomatoes and courgettes, flowers such as broccoli, and seeds such as pulses.

| Vegetable | | | |
|---|---|---|---|
| Species | Parts used | Origin | Cultivars |
| Brassica oleracea | leaves, axillary buds, | Europe | cabbage, Brussels |
| | stems, flower heads | | sprouts, cauliflower, broccoli, kale, |
| | | | kohlrabi, |
| | | | red cabbage, Savoy |
| | | | cabbage, Chinese broccoli, collard |
| | | | greens |
| Brassica rapa | root, leaves | Asia | turnip, Chinese cabbage, napa |
| | | | cabbage, bok choy |
| Raphanus sativus | root, leaves, seed | Southeastern Asia | radish, daikon, seedpod varieties |
| | pods, seed oil, | | |
| | sprouting | | |
| Daucus carota | root, leaves, stems | Persia | carrot |
| Pastinaca sativa | root | Eurasia | parsnip |
| Beta vulgaris | root, leaves | Europe and Near | beetroot, sea beet, Swiss |
| | | East | chard, sugar beet |
| Lactuca sativa | leaves, stems, seed | Egypt | lettuce, celtuce |
| | oil | | |
| Phaseolus vulgaris | pods, seeds | Central and South | green bean, French bean, runner |
| Phaseolus coccineus | | America | bean, haricot bean, Lima bean |
| Phaseolus lunatus | | | |
| Vicia faba | pods, seeds | Mediterranean and | broad bean |
| | | Middle East | |
| Pisum sativum | pods, seeds, sprouts | Mediterranean and | pea, snap pea, snow pea, split pea |
| | | Middle East | |
| Solanum tuberosum | tubers | South America | potato |
| Solanum melongena | fruits | South and East | eggplant (aubergine) |
| | | Asia | |
| Solanum | fruits | South America | tomato |
| lycopersicum | | | |
| Cucumis sativus | fruits | Southern Asia | cucumber |
| Cucurbita spp. | fruits, flowers | Mesoamerica | pumpkin, squash, marrow, zucchini |
| | | | (courgette), gourd |
| Allium cepa | bulbs, leaves | Asia | onion, spring onion, scallion, shallot |
| Allium sativum | bulbs | Asia | garlic |
| Allium | leaf sheaths | Europe and | leek, elephant garlic |
| ampeloprasum | | Middle East | |
| Capsicum annuum | fruits | North and South | pepper, bell pepper, sweet pepper |
| | | America | |
| Spinacia oleracea | leaves | Central and | spinach |
| | | southwestern Asia | |
| Dioscorea spp. | tubers | Tropical Africa | yam |
| Ipomoea batatas | tubers, leaves, shoots | Central and South | sweet potato |
| | | America | |
| Manihot esculenta | tubers | South America | cassava |

As stated previously, it is believed, without intending to be bound thereby, that the method of the instant claimed invention could be used in the cultivation of all fruits and vegetables.

It is also believed, without intending to be bound thereby, that the method of the instant claimed invention could be used in the cultivation of plants such as tobacco and Cannabis and Hemp.

It is also believed, without intending to be bound thereby, that the method of the instant claimed invention could be used in the cultivation of trees, including but not limited to trees harvested for use as Christmas Trees.

### Examples

A grower has 15 grow rooms of cannabis plants in a variety of growth states including vegetative and flower stages. The appropriate fertigation solution (shown below) is fed to each room, along with irrigation water (no nutrients) and collected in a large tank.

Flower Stage

| | Amount of | | |
|---|---|---|---|
| | each | | |
| | nutrient | ppm Tracer | Tracer |
| N (NO₃_{^{−}}) | 141.375 | 0.141375 | PTSA |
| K | 433 | 0.0433 | Rhodamine |
| P | 96 | 0.096 | NDSA |
| Mg | 120 | | |
| Ca | 186.537 | | |
| S | 158.337 | | |
| Fe | 6 | | |
| Zn | 0.3 | | |
| B | 1 | | |
| Cu | 0.3 | | |
| Mo | 0.2 | | |
| Na | 1.159 | | |
| Si | 0 | | |
| Cl | 0 | | |
| Mn | 1 | | |
| N (NH₄_{⁺}) | 10.799 | | |
| EC | 2.496 mS/cm | | |

Vegetative Stage

| Formula | Veg | ppm Tracer | Tracer |
|---|---|---|---|
| N (NO₃_{^{−}}) | 117.357 | 0.117357 | PTSA |
| K | 324 | 0.0324 | Rhodamine |
| P | 73 | 0.073 | NDSA |
| Mg | 100 | | |
| Ca | 154.846 | | |
| S | 131.947 | | |
| Fe | 4.29 | | |
| Zn | 0.23 | | |
| B | 1.5 | | |
| Cu | 0.28 | | |
| Mo | 0.05 | | |
| Na | 0.024 | | |
| Si | 0 | | |
| Cl | 0 | | |
| Mn | 0.72 | | |
| N (NH₄_{⁺}) | 8.965 | | |
| EC | 2.081 mS/cm | | |

Flower

| | | Ppm | |
|---|---|---|---|
| | Mix | Tracer | Tracer |
| N (NO₃_{^{−}}) | 60.22 | 0.06 | PTSA |
| K | 180.5 | 0.018 | Rhodamine |
| P | 40.15 | 0.04 | NDSA |
| Mg | 51.16 | | |
| Ca | 79.46 | | |
| S | 67.5 | | |
| Fe | 2.48 | | |
| Zn | 0.13 | | |
| B | 0.5 | | |
| Cu | 0.13 | | |
| Mo | 0.07 | | |
| Na | 0.39 | | |
| Si | 0 | | |
| Cl | 0 | | |
| Mn | 0.41 | | |
| N (NH₄_{⁺}) | 4.6 | | |
| E.C. | 1.7 mS/cm | | |

Based solely on the E.C. measurement it is impossible to tell what levels of either macros or micros are available. However, since the instant claimed invention measures the tracer levels, the solution to a process with either too much or too little of a nutrient in either a Vegetative or Flower based formula may be modified through dilution, when there is too much nutrient present, or adding nutrient when there is too little nutrient present.

### Strawberry Plant in the Fruiting Stage

| | Plant | | | | |
|---|---|---|---|---|---|
| | Strawberry | | | Ppm | |
| | Concentration | | ppm | Tracer | |
| Element | (ppm) | Fruiting | Tracer | (fruiting) | Tracer |
| N (NO₃_{^{−}}) | 80 | 128 | 0.08 | 0.128 | PTSA |
| N (NH₄_{⁺}) | 0 | | | | |
| P | 45 | 58 | 0.045 | 0.058 | NDSA |
| K | 100 | 211 | 0.01 | 0.0211 | Rhodamine |
| | | | | | WT |
| Mg | 50 | 40 | | | |
| Ca | 200 | 104 | | | |
| S | 180 | 54 | | | |
| Fe | 3 | 5 | | | |
| Zn | 0.5 | 0.25 | | | |
| B | 0.5 | 0.7 | | | |
| Mn | 0.5 | 2 | | | |
| Cu | 0.05 | 0.07 | | | |
| Mo | 0.05 | 0.05 | | | |

### Lettuce Plant

Plant

| | | ppm | |
|---|---|---|---|
| Element | Lettuce | Tracer | Tracer |
| N (NO₃_{^{−}}) | 165 | 0.165 | PTSA |
| N (NH₄_{⁺}) | 15 | 0.015 | PTSA |
| P | 50 | 0.05 | NDSA |
| K | 210 | 0.021 | Rhodamine |
| | | | WT |
| Mg | 45 | | |
| Ca | 190 | | |
| S | 65 | | |
| Fe | 4 | | |
| Zn | 0.1 | | |
| B | 5 | | |
| Mn | 0.5 | | |
| Cu | 0.1 | | |
| Mo | 0.05 | | |

### Pepper Plant

Plant

| | | ppm | |
|---|---|---|---|
| Element | Pepper | Tracer | Tracer |
| N (NO₃_{^{−}}) | 190 | 0.190 | PTSA |
| N (NH₄_{⁺}) | 18 | 0.018 | PTSA |
| P | 40 | 0.04 | NDSA |
| K | 340 | 0.0340 | Rhodamine |
| | | | WT |
| Mg | 50 | | |
| Ca | 170 | | |
| S | 360 | | |
| Fe | 5 | | |
| Zn | 0.33 | | |
| B | 0.33 | | |
| Mn | 0.55 | | |
| Cu | 0.05 | | |
| Mo | 0.05 | | |

### Tomato Plant

| | To- | To- | To- | | | | |
|---|---|---|---|---|---|---|---|
| | mato | mato | mato | ppm | Ppm | Ppm | |
| | stage | Stage | Stage | Trac- | Trac- | Trac- | |
| | 1-10 | 2- | 3 | er | er | er | |
| | to 14 | first | to plant | Stage | Stage | Stage | |
| Plant | days | cluster | maturity | 1 | 2 | 3 | Tracer |
| Element | | | | | | | |
| N | 100 | 130 | 180 | 0.100 | 0.130 | 0.180 | PTSA |
| (NO₃⁻) | | | | | | | |
| N | | | | | | | |
| (NH₄⁺) | | | | | | | |
| P | 40 | 55 | 65 | 0.04 | 0.055 | 0.065 | NDSA |
| K | 200 | 300 | 400 | 0.02 | 0.03 | 0.04 | Rhodamine |
| | | | | | | | WT |
| Mg | 20 | 33 | 45 | | | | |
| Ca | 100 | 150 | 400 | | | | |
| S | 53 | 109 | 144 | | | | |
| Fe | 3 | 3 | 3 | | | | |
| Zn | 0.1 | 0.1 | 0.1 | | | | |
| B | 0.3 | 0.3 | 0.3 | | | | |
| Mn | 0.8 | 0.8 | 0.8 | | | | |
| Cu | 0.07 | 0.07 | 0.07 | | | | |
| Mo | 0.03 | 0.03 | 0.03 | | | | |

In this case it may be more useful to track Ca levels versus P levels as the P levels are held relatively constant. Substituting NDSA at 0.1 ppm NDSA per 100 ppm of Calcium yields the following.

| | To- | To- | To- | | | | |
|---|---|---|---|---|---|---|---|
| | mato | mato | mato | | | | |
| | stage | Stage | Stage | ppm | Ppm | Ppm | |
| | 1-10 | 2- | 3 to | Tracer | Tracer | Tracer | |
| | to 14 | first | plant | Stage | Stage | Stage | |
| Plant | days | cluster | maturity | 1 | 2 | 3 | Tracer |
| Element | | | | | | | |
| N | 100 | 130 | 180 | 0.100 | 0.130 | 0.180 | PTSA |
| (NO₃⁻) | | | | | | | |
| N | | | | | | | |
| (NH₄⁺) | | | | | | | |
| P | 40 | 55 | 65 | | | | |
| K | 200 | 300 | 400 | 0.02 | 0.03 | 0.04 | Rhodamine |
| | | | | | | | WT |
| Mg | 20 | 33 | 45 | | | | |
| Ca | 100 | 150 | 400 | 0.1 | 0.15 | 0.4 | NDSA |
| S | 53 | 109 | 144 | | | | |
| Fe | 3 | 3 | 3 | | | | |
| Zn | 0.1 | 0.1 | 0.1 | | | | |
| B | 0.3 | 0.3 | 0.3 | | | | |
| Mn | 0.8 | 0.8 | 0.8 | | | | |
| Cu | 0.07 | 0.07 | 0.07 | | | | |
| Mo | 0.03 | 0.03 | 0.03 | | | | |

### Cannabis

| | Plant | | | | |
|---|---|---|---|---|---|
| | Cannabis | Cannabis | | Ppm | |
| | (Vegetative | (Flowering | ppm Tracer | Tracer | |
| | Stage) | Stage) | (Vegetative) | (flowering) | Tracer |
| N (NO₃_{^{−}}) | 160 | 140 | 0.16 | 0.14 | PTSA |
| N (NH₄_{⁺}) | 0 | 0 | | | |
| P | 30 | 80 | 0.03 | 0.08 | NDSA |
| K | 180 | 210 | 0.018 | 0.021 | Rhodamine |
| | | | | | WT |
| Mg | 36 | 90 | | | |
| Ca | 176 | 176 | | | |
| S | 67 | 147 | | | |
| Fe | 2 | 1.5 | | | |
| Zn | 0.23 | 0.2 | | | |
| B | 1 | 1 | | | |
| Mn | 0.35 | 0.35 | | | |
| Cu | 0.21 | 0.21 | | | |
| Mo | 0.15 | 0.15 | | | |

In addition, the tracing can also be used to dilute a leachate stream prior to being reused as fertigation. In some cases, the leachate will be more concentrated than the fertigation stream so the leachate must be diluted with suitable water (typically RO or dehumidification condensate) before it is processed further.

| | | | | | | Minimum |
|---|---|---|---|---|---|---|
| | | | | | | Necessary |
| | | | Cannabis | ppm | | Dilution |
| | | | Vegetative | Tracer | | for |
| | Cannabis | ppm | tank | (Vege- | | Leachate |
| Plant | Leachate | Tracer | targets) | tative) | Tracer | NPK |
| N | 240 | 0.24 | 180 | 0.18 | PTSA | 25% |
| (NO₃⁻) | | | | | | |
| N | 0 | | 0 | | | |
| (NH₄⁺) | | | | | | |
| P | 78 | 0.078 | 70 | 0.07 | NDSA | 10% |
| K | 329 | 0.0329 | 260 | 0.026 | Rhodamine | 21% |
| | | | | | WT | |
| Mg | 165 | | 107 | | | |
| Ca | 295 | | 226 | | | |
| S | 233 | | 137 | | | |
| Fe | 3.22 | | 4.06 | | | |
| Zn | 0.27 | | 0.24 | | | |
| B | 8.11 | | 6.92 | | | |
| Mn | 0.27 | | 0.35 | | | |
| Cu | 0.29 | | 0.25 | | | |
| Mo | 0.06 | | 0.12 | | | |

Note that in this case dilution of the system will result in higher S levels versus the target. Dilution of the water by 41% would be required to meet the S levels and corresponding Mg levels (assuming MgSO₄ is the source).

Tracing the S in this situation is more useful.

| | | | | | | Mini- |
|---|---|---|---|---|---|---|
| | | | | | | mum |
| | | | | | | Neces- |
| | | | Cannabis | | | sary |
| | | | Vege- | ppm | | Dilution |
| | | | tative | Tracer | | for |
| | Cannabis | ppm | tank | (Vege- | | Leachate |
| Plant | Leachate | Tracer | targets) | tative) | Tracer | NPKS |
| N | 240 | 0.24 | 180 | 0.18 | PTSA | 25% |
| (NO₃⁻) | | | | | | |
| N | 0 | | 0 | | | |
| (NH₄⁺) | | | | | | |
| P | 78 | | 70 | | | |
| K | 329 | 0.0329 | 260 | 0.026 | Rhodamine | 21% |
| | | | | | WT | |
| Mg | 165 | | 107 | | | |
| Ca | 295 | | 226 | | | |
| S | 233 | 0.233 | 137 | 0.137 | NDSA | 41% |
| Fe | 3.22 | | 4.06 | | | |
| Zn | 0.27 | | 0.24 | | | |
| B | 8.11 | | 6.92 | | | |
| Mn | 0.27 | | 0.35 | | | |
| Cu | 0.29 | | 0.25 | | | |
| Mo | 0.06 | | 0.12 | | | |

Alternatively, a fourth tracer could be used for P as well.

| | | | | | | Mini- |
|---|---|---|---|---|---|---|
| | | | | | | mum |
| | | | Can- | | | Neces- |
| | | | nabis | | | sary |
| | | | Vege- | ppm | | Dilution |
| | Can- | | tative | Tracer | | for |
| | nabis | ppm | tank | (Vege- | | Leachate |
| Plant | Leachate | Tracer | targets) | tative) | Tracer | NPKS |
| N | 240 | 0.24 | 180 | 0.18 | PTSA | 25% |
| (NO₃⁻) | | | | | | |
| N | 0 | | 0 | | | |
| (NH₄⁺) | | | | | | |
| P | 78 | 0.078 | 70 | 0.07 | 2- | 10% |
| | | | | | anthracene- | |
| | | | | | sulfonic | |
| | | | | | acid | |
| K | 329 | 0.0329 | 260 | 0.026 | Rhodamine | 21% |
| | | | | | WT | |
| Mg | 165 | | 107 | | | |
| Ca | 295 | | 226 | | | |
| S | 233 | 0.233 | 137 | 0.137 | NDSA | 41% |
| Fe | 3.22 | | 4.06 | | | |
| Zn | 0.27 | | 0.24 | | | |
| B | 8.11 | | 6.92 | | | |
| Mn | 0.27 | | 0.35 | | | |
| Cu | 0.29 | | 0.25 | | | |
| Mo | 0.06 | | 0.12 | | | |

Once the leachate has been diluted properly the remaining components can be adjusted using the fluorescent signals.

1. A method to optimize horticulture of growing plants by determining whether the desired amount of at least two nutrient products is present in water, wherein the water is used to deliver nutrients to growing plants; comprising the steps of:
a) adding a known amount of an inert fluorescent tracer to a known amount of a nutrient, wherein said nutrient is useful to provide nutrition to growing plants;
b) adding at least two or more mixtures of inert fluorescent tracer and nutrient to water to create a nutrient liquid; wherein the two or more mixtures have different fluorescent tracers and a different nutrient;
c) using two or more fluorometers to determine the concentration of two or more inert fluorescent materials in the water and using the concentration of two or more inert fluorescent tracers to calculate the amount of each nutrient present in the water;
d) adjusting the amount of nutrient present in the water when at least one of the fluorescent signals of at least one of the inert fluorescent tracers indicates either too much or too little of the nutrient matched with that inert fluorescent tracer is present; and
e) applying the nutrient liquid to growing plants.

2. The method of claim 1, wherein the inert fluorescent material is selected from the group consisting of:
1,3,6,8-pyrenetetrasulfonic acid, tetrasodium salt (CAS Registry No. 59572-10-0);
1,5-naphthalenedisulfonic acid, disodium salt (hydrate) (CAS Registry No. 1655-29-4, aka 1,5-NDSA hydrate);
xanthylium, 9-(2,4-dicarboxyphenyl)-3,6-bis(diethylamino), chloride, disodium salt, also known as Rhodamine WT (CAS Registry No. 37299-86-8);
C.I. Fluorescent Brightener 230, also known as Leucophor BSB (CAS Registry No. 68444-86-0);
benzenesulfonic acid, 2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Leucophor BMB (CAS Registry No. 16470-249, aka Leucophor U, Flu. Bright. 290);
9,9′-biacridinium, 10,10′-dimethyl-, dinitrate, also known as Lucigenin (CAS Registry No. 2315-97-1, aka bis-N-methylacridinium nitrate);
1-deoxy-1-(3,4-dihydro-7,8-dimethyl-2,4-dioxobenzo[g]pteridin-10(2H)-yl)-D-ribitol, also known as Riboflavin or Vitamin B2 (CAS Registry No. 83-88-5);
3,6-acridinediamine, N,N,N′,N′-tetramethyl-, monohydrochloride, also known as Acridine Orange (CAS Registry No. 65-61-2);
2-anthracenesulfonic acid sodium salt (CAS Registry No. 16106-40-4);
1,5-anthracenedisulfonic acid (CAS Registry No. 61736-91-2) and salts thereof;
2,6-anthracenedisulfonic acid (CAS Registry No. 61736-95-6) and salts thereof;
1,8-anthracenedisulfonic acid (CAS Registry No. 61736-92-3) and salts thereof;
anthra[9,1,2-cde]benzo[rst]pentaphene-5,10-diol,16,17-dimethoxy-,bis(hydrogen sulfate), disodium salt, also known as Anthrasol Green IBA (CAS Registry No. 2538-84-3, aka Solubilized Vat Dye);
bathophenanthrolinedisulfonic acid disodium salt (CAS Registry No. 52746-49-3);
amino 2,5-benzene disulfonic acid (CAS Registry No. 41184-20-7);
2-(4-aminophenyl)-6-methylbenzothiazole (CAS Registry No. 92-364);
1H-benz[de]isoquinoline-5-sulfonic acid, 6-amino-2,3-dihydro-2-(4-methylphenyl)1,3-dioxo-, monosodium salt, also known as Brilliant Acid Yellow 8G (CAS Registry No. 2391-30-2, aka Lissamine Yellow FF, Acid Yellow 7);
phenoxazin-5-ium, 1-(aminocarbonyl)-7-(diethylamino)-3,4-dihydroxy-, chloride, also known as Celestine Blue (CAS Registry No. 1562-90-9);
benzo[a]phenoxazin-7-ium, 5,9-diamino-, acetate, also known as cresyl violet acetate (CAS Registry No. 10510-54-0);
4-dibenzofuransulfonic acid (CAS Registry No. 42137-76-8);
3-dibenzofuransulfonic acid (CAS Registry No. 215189-98-3);
1-ethylquinaldinium iodide (CAS Registry No. 606-53-3);
fluorescein (CAS Registry No. 2321-07-5);
fluorescein, sodium salt (CAS Registry No. 518-47-8, aka Acid Yellow 73, Uranine);
Keyfluor White ST (CAS Registry No. 144470-48-4, aka Flu. Bright 28);
Benzenesulfonicacid2,2′-(1,2-ethenediyl)bis[5-[[4-[bis(2-hydroxyethyl)amino]-6-[(4-sulfophenyl)amino]-1,3,5-triazin-2-yl]amino]-, tetrasodium salt, also known as Keyfluor White CN (CAS Registry No. 16470-24-9);
pyranine, (CAS Registry No. 6358-69-6, aka 8-hydroxy-1, 3, 6-pyrenetrisulfonic acid, trisodium salt);
quinoline (CAS Registry No. 91-22-5);
3H-phenoxazin-3-one, 7-hydroxy-, 10-oxide, also known as Rhodalux (CAS Registry No. 550-82-3);
xanthylium, 9-(2,4-dicarboxyphenyl)-3,6-bis(diethylamino)-, chloride, disodium salt, also known as Rhodamine WT (CAS Registry No. 37299-86-8); and
phenazinium, 3,7-diamino-2,8-dimethyl-5-phenyl-, chloride, also known as Safranine 0 (CAS Registry No. 477-73-6).

3. The method of claim 2 in which the inert fluorescent tracer is selected from the group consisting of 1,3,6,8-pyrenetetrasulfonic acid, tetrasodium salt (CAS Registry No. 59572-10-0, aka PTSA); 1,5-naphthalenedisulfonic acid, disodium salt (hydrate) (CAS Registry No. 1655-29-4, also known as 1,5-NDSA hydrate); and
xanthylium, 9-(2,4-dicarboxyphenyl)-3,6-bis(diethylamino), chloride, disodium salt, also known as Rhodamine WT (CAS Registry No. 37299-86-8).

4. The method of claim 1, wherein the nutrients present in the nutrient liquid include nitrogen, phosphorus, and potassium.

5. The method of claim 3 in which the inert fluorescent tracer for nitrogen is PTSA, the inert fluorescent tracer for phosphorus is NDSA and the inert fluorescent tracer for potassium is Rhodamine.

6. The method of claim 1 wherein the Horticulture methods are selected from the group consisting of Traditional Horticulture, Hydroponics, Passive hydroponics, Hydroculture, Deep water culture, Top-fed deep water culture, Rotary Hydroponic Garden, Inorganic hydroponic solutions, Organic hydroponic solutions, hydroponic solutions, Aeroponics, Fogponics and Fertigation.

7. The method of claim 1 wherein the growing plants are selected from the group consisting of fruits, vegetables, tobacco, Cannabis, Hemp and trees.

8. The method of claim 7 wherein the growing plants are selected from the group consisting of fruits.

9. The method of claim 7 wherein the growing plants are selected from the group consisting of vegetables.

10. The method of claim 7 wherein the growing plants are selected from the group consisting of tobacco, Cannabis and Hemp Plants.

11. The method of claim 7 wherein the growing plants are tobacco.

12. The method of claim 7 wherein the growing plants are Cannabis.

13. The method of claim 7 wherein the growing plants are Hemp.

14. The method of claim 7 wherein the growing plants are trees.
